(19) United States
(12) Patent Application Publication
(10) Pub. No.: US 2026/0294614 A1
(43) Pub. Date: Oct. 1, 2026

(54) LIFTING LINE WITH SPECIAL-SHAPED CROSS-SECTION

(71) Applicant: DIAMOND BIOTECHNOLOGY CO., LTD., New Taipei City (TW)

(72) Inventor: CHIA HSIEN HSIEH, New Taipei City (TW)

(21) Appl. No.: 19/096,663

(22) Filed: Mar. 31, 2025

(51) Int. Cl. A61F 2/10

(52) U.S. Cl. CPC A61F 2/105; A61F 2220/0016

(57) ABSTRACT

A lifting line with special-shaped cross-section is provided, including a wire having a cross-section having a special-shaped structure; a plurality of barbs, the plurality of barbs arranged on the wire, the barbs including at least one planar surface; and a plurality of grooves arranged adjacent to one side of the barbs. The contact area of the wire is increased, and the friction force can be distributed more evenly, thereby reducing local friction pressure and improving durability. Additionally, the special-shaped structure increases the contact area with the skin tissue, enhances friction, reduces the sliding of the lifting line in the skin, and helps to improve the stability of the lifting line during tightening or stretching, making it more difficult to loosen. Such a design helps to enhance the stability of the suture and ensures that the sutures are tighter, thereby effectively avoiding problems such as uneven sutures or excessive gap.

### BACKGROUND OF THE INVENTION

### (a) Technical Field of the Invention

The present invention relates generally to a lifting line, and more particularly to a lifting line having a special-shaped cross-section.

### (b) Description of the Prior Art

Lifting line is a modern cosmetic medicine technology, commonly used in facial anti-aging treatments, and is a non-surgical facial lifting method, usually used to improve facial sagging and other problems, and to achieve the effects of lifting, firming and shaping. The lifting line is a specially made absorbable medical suture, usually made of biocompatible materials, such as polydioxanone (PDO). After inserted into the skin, the lifting line will be gradually absorbed by the body, and will stimulate the skin tissue to produce collagen, during the course of absorption, thereby helping to improve the elasticity and firmness of the skin.

For example, Taiwan Utility Model M580945 discloses a “suture for plastic surgery”, which includes a wire and multiple sharp protrusions (such as barbs). The surface of the wire is cut in the same or opposite directions within a spiral angle through a double-knife cutting operation, so that multiple grooves are formed in the surface of the wire. After the cutting of each groove, a sharp protrusion for pulling the skin is formed to correspond thereto in a one-by-one manner.

However, M580945 teaches, in FIGS. 1 and 2, the suture is cut on the surface of the wire by means of double-knife cutting so that multiple grooves and multiple sharp protrusions are formed on the wire. Since the grooves have a small length and are inclined, the grooves do not have enough space to get stuck in human tissue, so that it can only rely on sharp protrusions to grasp human tissues. However, sharp protrusions can easily injure human tissues and increase pain. Moreover, it can be seen from FIGS. 1 and 2 of M580945 that in order to have the sharp protrusions protrude from the surface of the wire to get erected, the cutting depth is almost half of the wire diameter, and the structure of the wire is severely damaged, making the structural strength relatively weak and easy to break. Since the suture only relies on the grooves and multiple sharp protrusions to grasp the skin tissue, the pull value is relatively poor. If improvement can be made on the structure to enhance the pull value, the lifting effect will be increased, so as to help fight against the skin sagging caused by the gravity and aging.

### SUMMARY OF THE INVENTION

To achieve the objective of the present invention, the present invention provides a lifting line with special-shaped cross-section, comprising: a wire, the wire having a cross-section having a special-shaped structure; a plurality of barbs, the plurality of barbs being arranged on the wire, the barbs comprising at least one planar surface; and a plurality of grooves, the plurality of grooves being arranged adjacent to one side of the barbs.

In the above, the special-shaped structure may be one of a multi-lobed shape, a three-lobed shape, and a triangular shape.

In the above, the plurality of barbs may be arranged on the wire in a linear or spiral manner.

In the above, the plurality of grooves may be arranged on the wire in a linear or spiral manner.

In the above, the plurality of barbs may be arranged on the wire in an equal spacing or an unequal spacing manner.

In the above, the plurality of grooves may be arranged on the wire in an equal spacing or an unequal spacing manner.

In the above, a distance between one of the barbs and an adjacent one of the barbs may be 0.5 mm-1.5 mm.

By providing the cross-section of the wire with a special-shaped structure, the contact area of the wire is increased, and the friction force can be distributed more evenly, thereby reducing local friction pressure and improving durability. Additionally, the special-shaped structure increases the contact area with the skin tissue, enhances friction, reduces the sliding of the lifting line in the skin, and helps to improve the stability of the lifting line during tightening or stretching, making it more difficult to loosen. Such a design helps to enhance the stability of the suture and ensures that the sutures are tighter, thereby effectively avoiding problems such as uneven sutures or excessive gaps.

### BRIEF DESCRIPTION OF THE DRAWINGS

FIG. 1 is a schematic view showing a wire according to the present invention having a cross-section having a multi-lobed shape.

FIG. 2 is a schematic view showing a wire according to the present invention having a cross-section having a multi-lobed shape and barbs arranged in a spiral manner.

FIG. 3 is a schematic view showing a wire according to the present invention having a cross-section having a multi-lobed shape and barbs arranged in a non-equal spacing manner.

FIG. 4 is a schematic view showing a wire according to the present invention having a cross-section having a three-lobed shape.

FIG. 5 is a schematic view showing a wire according to the present invention having a cross-section having a three-lobed shape and barbs arranged in a spiral manner.

FIG. 6 is a schematic view showing a wire according to the present invention having a cross-section having a three-lobed shape and barbs arranged in a non-equal spacing manner.

FIG. 7 is a schematic view showing a wire according to the present invention having a cross-section having a triangular shape.

FIG. 8 is a schematic view showing a wire according to the present invention having a cross-section having a triangular shape and barbs arranged in a spiral manner.

FIG. 9 is a schematic view showing a wire according to the present invention having a cross-section having a triangular shape and barbs arranged in a non-equal spacing manner.

FIG. 10 is a top plan view, in an enlarged form, showing a wire having a cross-section having a multi-lobed shape according to the present invention.

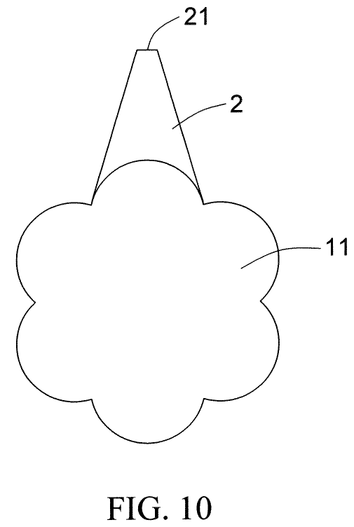

FIG. 11 is a top plan view, in an enlarged form, showing a wire having a cross-section having a three-lobed shape according to the present invention.

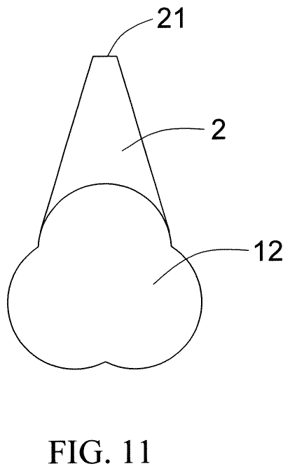

FIG. 12 is a top plan view, in an enlarged form, showing a wire having a cross-section having a triangular shape according to the present invention.

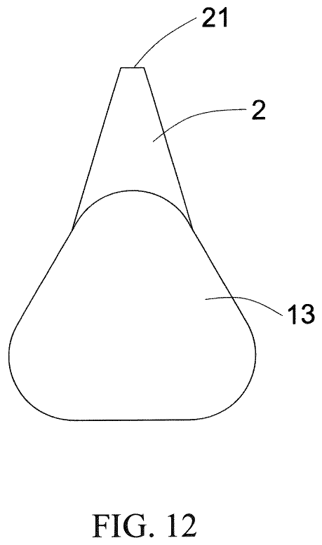

### DETAILED DESCRIPTION OF THE PREFERRED EMBODIMENTS

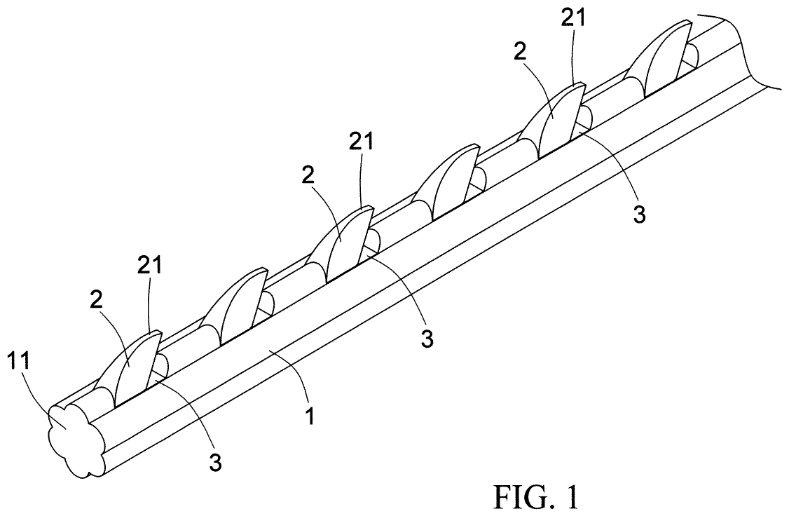

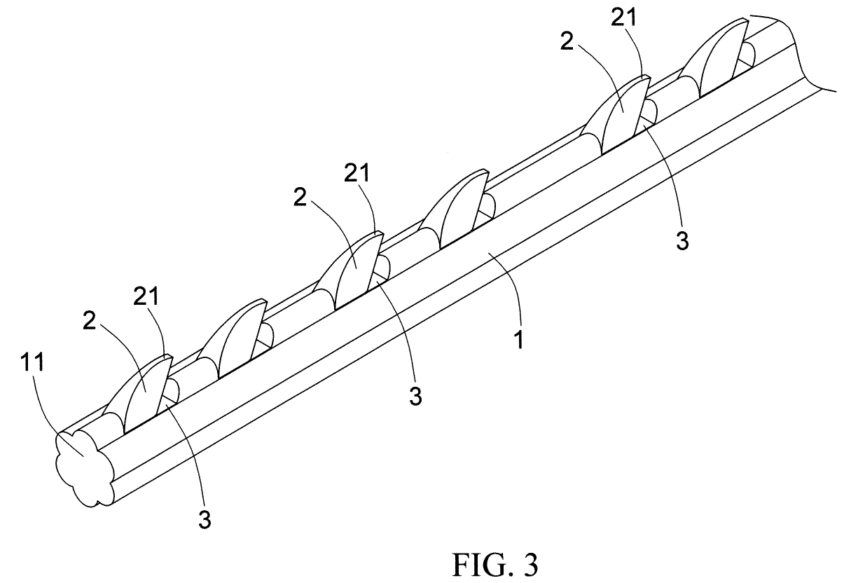

Referring to FIGS. 1, 2, and 3, the present invention provides a lifting line with special-shaped cross-section, comprising:
a wire 1, wherein the wire 1 has a cross-sectional surface having a special-shaped structure, wherein the wire 1 can be classified into absorbable or non-absorbable according to the material thereof, wherein the absorbable material is for example plain catgut, chromic gut, polyglycolic acid (such as Dexon), polyglycol lactic acid (such as Vicryl), polycaprolactone (PCL), polypropylene carbonate (PPC), polytrimethylene carbonate, polydioxanone (PDO), polyglycolide (PGA), poly(lactic-co-glycolic) acid (PLGA), lactide (LA), and polydioxanone (PDS), and the non-absorbable material is for example silk, cotton, nylon, polyester, polyethylene, polypropylene, polytetrafluoroethylene, stainless steel, and polybutylene, wherein the surface of the wire 1 can be coated with a material for prompting skin repair, such as collagen, wherein the special-shaped structure can be a multi-lobed shape 11, wherein the multi-lobed shape 11 refers to a structure in which multiple lobes extend outward from the middle, and depending on the number of lobes, the multi-lobed shape 11 may be a four-lobed shape (cross shape), a five-lobed shape, a six-lobed shape, and so on, and looks like a plurality of lobes are combined into one structure, wherein the specific number of lobes may be determined according to actual needs;
a plurality of barbs 2, wherein the plurality of barbs 2 are arranged on the wire body 1, and the barbs 2 comprise at least one planar surface 21, wherein the barbs 2 are used to fix the skin tissue to prevent loosening due to excessive pulling force, wherein the barbs 2 can be manufactured using common processes on the market, such as cutting, press molding or injection molding, wherein the plurality of barbs 2 can be arranged on the wire 1 in a linear or spiral arrangement, wherein the plurality of barbs 2 can be arranged on the wire 1 in an equal spacing or an unequal spacing arrangement, wherein the distance between one barb 2 and an adjacent barb 2 is 0.5 mm to 1.5 mm, where the distance between the barb 2 and the adjacent barb 2 can be the distance between the centers of the two barbs 2; and
a plurality of grooves 3, wherein the plurality of grooves 3 are respectively arranged adjacent to one side of the barbs 2, wherein the plurality of grooves 3 can be arranged on the wire 1 in a linear or spiral arrangement, wherein the plurality of grooves 3 can be arranged on the wire 1 in an equal or unequal spacing arrangement.

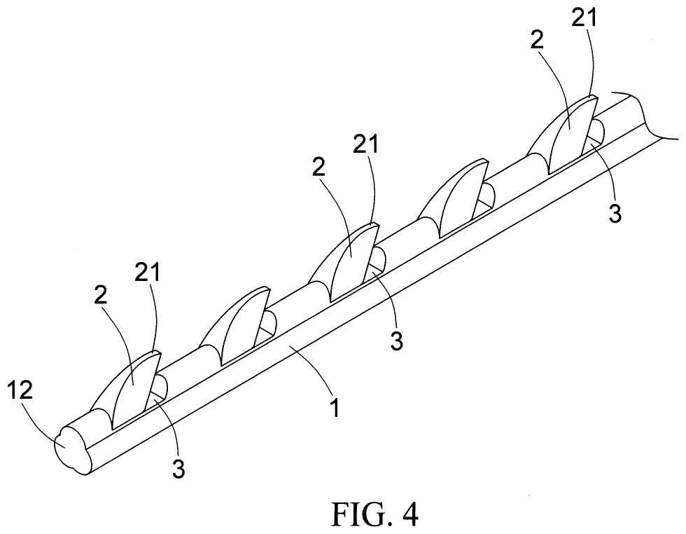

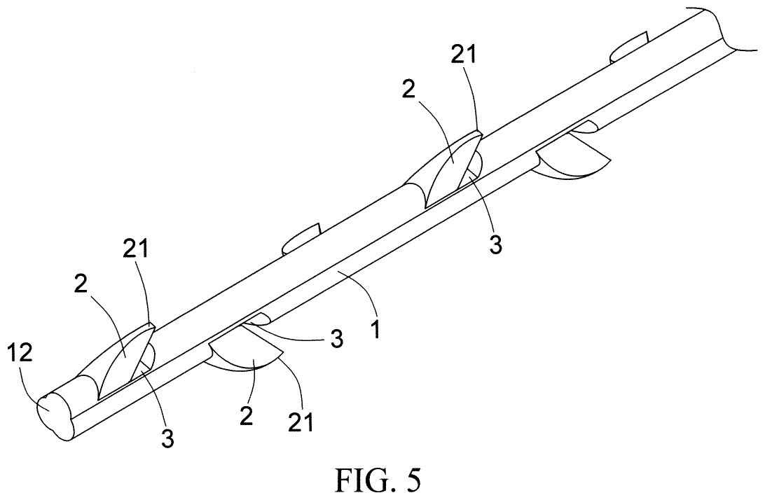

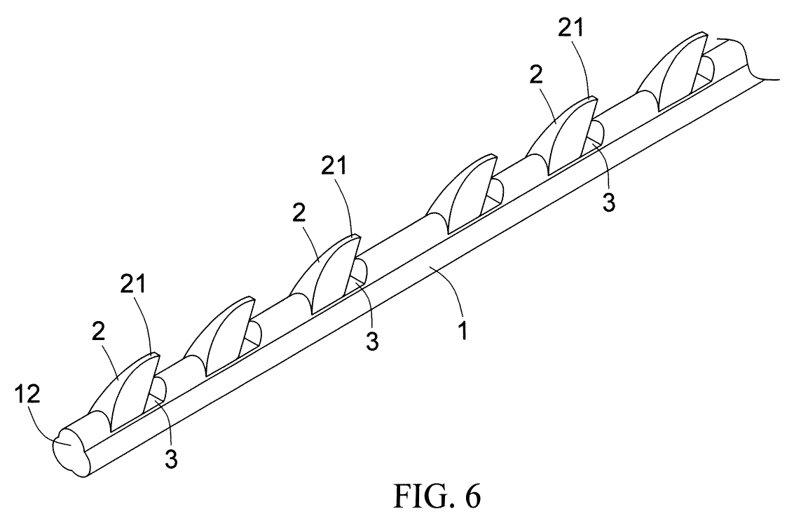

Referring to FIGS. 4, 5, and 6, the special-shaped structure can be a three-lobed shape 12. Similarly, the plurality of barbs 2 can be arranged on the wire 1 in a linear or spiral arrangement. The plurality of barbs 2 can be arranged on the wire 1 in an equal spacing or an unequal spacing arrangement. The plurality of grooves 3 can be arranged on the wire 1 in a linear or spiral arrangement. The plurality of grooves 3 can be arranged on the wire 1 in an equal spacing or an unequal spacing arrangement.

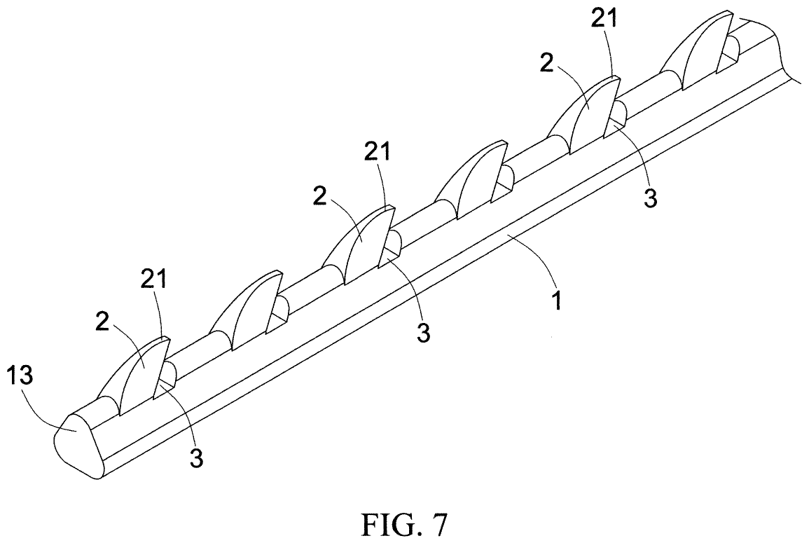

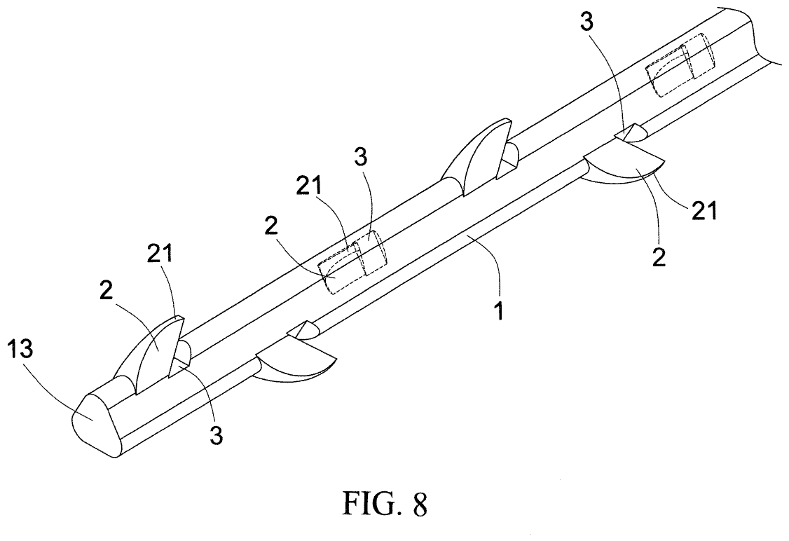

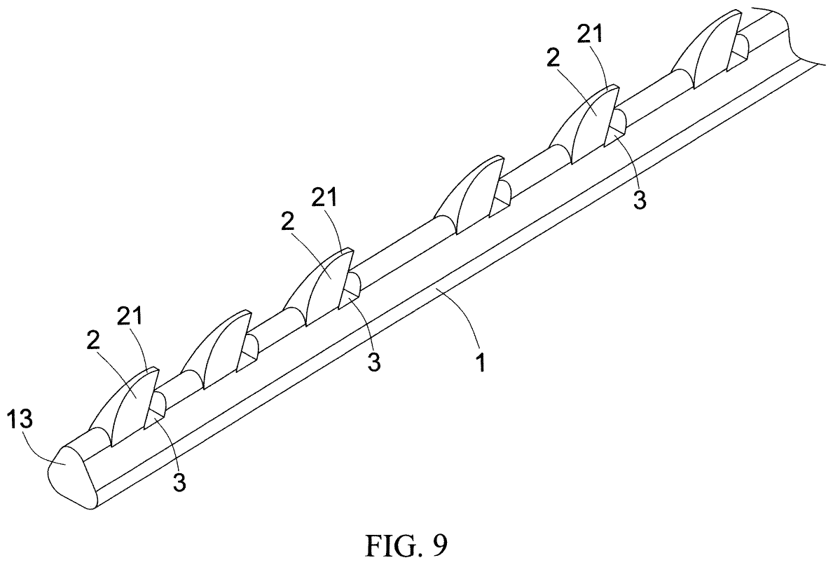

Referring to FIGS. 7, 8, and 9, the special-shaped structure can be a triangular shape 13. Similarly, the plurality of barbs 2 can be arranged on the wire 1 in a linear or spiral arrangement. The plurality of barbs 2 can be arranged on the wire 1 in an equal spacing or an unequal spacing arrangement. The plurality of grooves 3 can be arranged on the wire 1 in a linear or spiral arrangement. The plurality of grooves 3 can be arranged on the wire 1 in an equal spacing or an unequal spacing arrangement.

In addition to the multi-lobed shape 11, the three-lobed shape 12, and the triangular shape 13, the special-shaped structure can also be a polygonal shape, a cross shape, a flat shape, a Y shape, an H shape or a dumbbell shape. The polygonal shape refers to a shape of which the structure has multiple angles. The number of angles and sides of the polygonal shape is not fixed and can be determined according to actual needs. The dumbbell shape refers to a dumbbell-like shape, usually with two round or spherical ends connected by a slender connecting part in the middle to form a dumbbell-like shape.

The lifting lines with special-shaped cross-section according to the present invention are tested separately, and the method and data are as follows:

### Method of Lifting Test

The lifting test is used to measure the maximum force required when an object (in this case, the lifting line) is pulled out or removed from a fixed base (in this case, pig skin tissue being used to replace human skin tissue). The lifting line is set to penetrate through the pig skin tissue to be implanted in an area to be barbed. After implantation, an excess length of the lifting line is removed and the pig skin tissue is clamped in a tensile testing machine to test the pull value, of which the unit is Newton (N).

### Data Result of Lifting Test

A pure cutting line (only including barbs and grooves) is used as a reference group, and various conditions for various special-shaped structures are used as experimental groups for testing lifting lines.

TABLE 1

| | Reference | | | | |
|---|---|---|---|---|---|
| | Group/Pure | Experimental | Experimental | Experimental | Experimental |
| Group | Cutting Line | Group A | Group B | Group C | Group D |
| Distance | 1.0 | 1.0 | 1.0 | 1.0 | 1.0 |
| (mm) | | | | | |
| Rotation | 45 | 45 | 90 | 45 | 30 |
| angle (°) | | | | | |
| special- | no special- | multi-lobed | multi-lobed | three-lobed | triangular |
| shaped | shaped | shape | shape | shape | shape |
| structure | structure | (six-lobed | (six-lobed | | |
| | | shape) | shape) | | |
| Pull | 3.7 | 5.1 | 4.7 | 4.5 | 3.9 |
| value | | | | | |
| (N) | | | | | |

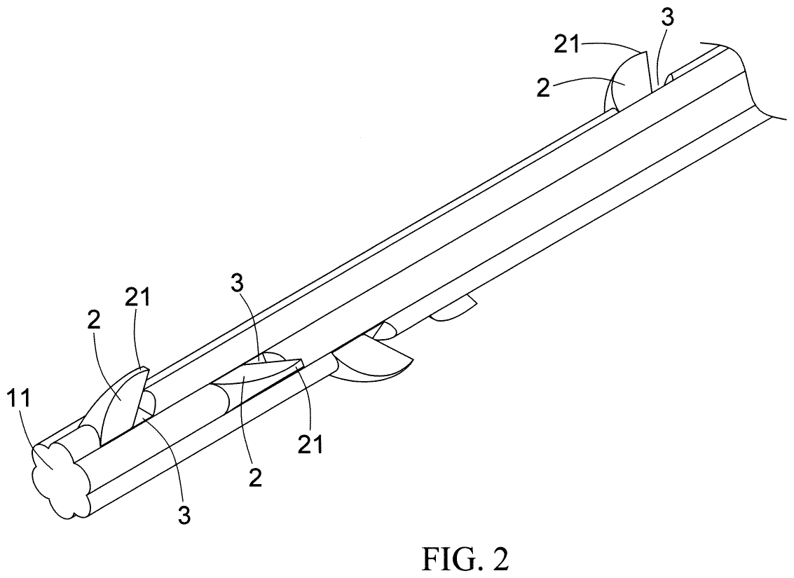

In Table 1, “distance” refers to the spacing distance between one barb 2 and an adjacent barb 2 (see for example FIG. 1), and the unit is millimeter (mm). “Rotation angle” refers to the angle of rotation of the barbs 2 at every certain distance for the barbs 2 being arranged in a spiral manner on the wire body 1 (see for example FIG. 2), and the unit is degree) (°. The special-shaped structure refers to the multi-lobed shape 11 shown in FIG. 1, the three-lobed shape 12 shown in FIG. 4, and the triangular shape 13 shown in FIG. 7.

It can be seen from Table 1 that the pull values of Experimental Group A, Experimental Group B, Experimental Group C, and Experimental Group D are all greater than that of the Reference Group. A larger pull value indicates the lifting line can withstand a greater force during the lifting process without being pulled out or falling off.

A larger pull value indicates:
(1) Stronger fixation: The lifting line can be more firmly fixed in the skin tissue, reducing the possibility of falling off or sliding;
(2) Higher pull resistance: The lifting line can resist greater pull forces or external forces, providing more stable and longer-lasting effects during use; and
(3) Better durability: A higher pull value may mean that the lifting line can keep in the fixed state and avoid premature failure or damage under repeated stretching or stress.

Thus, compared with lifting lines without special-shaped structure, the lifting line with special-shaped structure can provide a better lifting effect.

Comparison between Experimental Group A and Reference Group shown in Table 1, it is found that the special-shaped structure having a multi-lobed shape may increase the effect by 20%-40%, that is 5.1−3.7/3.7=37.8%.

Compared with the prior art, the present invention has the following development advantages:

(1) The barbs 2 comprise at least one planar surface 21, so that the barbs 2 are not sharp and this effectively reduce the chance of puncture.

(2) The wire 1 is provided with grooves 3, allowing the skin tissue to be clamped in the grooves 3, so as to have the skin tissue clamped more firmly.

(3) By providing the cross-section of the wire 1 with a special-shaped structure, the contact area of the wire 1 is increased, and the friction force can be distributed more evenly, thereby reducing local friction pressure and improving durability. Additionally, the special-shaped structure increases the contact area with the skin tissue, enhances friction, reduces the sliding of the lifting line in the skin, and helps to improve the stability of the lifting line during tightening or stretching, making it more difficult to loosen. Such a design helps to enhance the stability of the suture and ensures that the sutures are tighter, thereby effectively avoiding problems such as uneven sutures or excessive gaps. Frictional pressure refers to the pressure generated by the contact surface between the lifting line and the skin tissue. When the lifting line contacts the skin tissue and moves relative to it, friction will be generated. This friction will act on the contact surface, thereby generating friction pressure. A greater friction pressure usually means a greater normal force per unit area, which increases frictional energy consumption. However, excessive friction pressure may cause excessive friction, which may affect the lifting effect and may even cause discomfort to the skin.

I claim:

1. A lifting line with special-shaped cross-section, comprising:
a wire, the wire having a cross-section having a special-shaped structure;
a plurality of barbs, the plurality of barbs being arranged on the wire, the barbs comprising at least one planar surface; and
a plurality of grooves, the plurality of grooves being arranged adjacent to one side of the barbs.

2. The lifting line with special-shaped cross-section according to claim 1, wherein the special-shaped structure comprises a multi-lobed shape, a three-lobed shape, or a triangular shape.

3. The lifting line with special-shaped cross-section according to claim 1, wherein the plurality of barbs are arranged on the wire in a linear or spiral arrangement.

4. The lifting line with special-shaped cross-section according to claim 1, wherein the plurality of grooves are arranged on the wire in a linear or spiral arrangement.

5. The lifting line with special-shaped cross-section according to claim 1, wherein the plurality of barbs are arranged on the wire in an equal spacing or an unequal spacing arrangement.

6. The lifting line with special-shaped cross-section according to claim 1, wherein the plurality of grooves are arranged on the wire in an equal spacing or an unequal spacing arrangement.

7. The lifting line with special-shaped cross-section according to claim 1, wherein a distance between one of the barbs and an adjacent one of the barbs is 0.5 mm-1.5 mm.
